(19) United States
(12) Patent Application Publication
(10) Pub. No.: US 2022/0022856 A1
(43) Pub. Date: Jan. 27, 2022

(54) OCCLUDER AND OCCLUDER FASTENING SYSTEM

(71) Applicant: HANGZHOU ENDONOM MEDTECH CO., LTD., Zhejiang (CN)

(72) Inventors: Yongsheng Wang, Zhejiang (CN); Xingzhi Liu, Zhejiang (CN)

(21) Appl. No.: 17/496,046

(22) Filed: Oct. 7, 2021

Related U.S. Application Data
(63) Continuation of application No. PCT/CN2020/084304, filed on Apr. 10, 2020.

(30) Foreign Application Priority Data
Apr. 11, 2019 (CN) 201910291642.4
Apr. 11, 2019 (CN) 201920489828.6

(51) Int. Cl. A61B 17/00; A61B 17/12

(52) U.S. Cl. CPC A61B 17/0057; A61B 17/12168; A61B 17/12122; A61B 2017/00632; A61B 2017/1205; A61B 2017/00623; A61B 2017/00606

(57) ABSTRACT

An occluder includes a proximal occlusion portion. The proximal occlusion portion has a proximal end provided with a first converged member, and is provided with a conical guide structure on an outer periphery of the first converged member. The guide structure has an inner diameter gradually decreasing from a distal end to a proximal end. The guide structure provides a guiding performance to facilitate the occluder to be withdrawn into the sheath easily. An occluder fastening system is used in cooperation with the occluder.

### TECHNICAL FIELD

The present invention relates to the technical field of medical devices, and in particular to an occluder and an occluder fastening system.

### BACKGROUND

Occluders, as implants for interventional therapy, are used in the treatment of atrial septal defect, ventricular septal defect and patent ductus arteriosus. The typical occluders include occluders for patent ductus arteriosus, occluders for atrial septal defect and occluders for ventricular septal defect. The occluder for patent ductus arteriosus has a basic structure employing a mushroom-shaped arrangement woven with a super-elastic nickel-titanium alloy, in which a polymer biofilm is attached therein, and the mesh structure is filled with polyester or polytetrafluoroethylene therein. The occluder for atrial septal defect and occluder for ventricular septal defect have a self-expanding two-disc structure densely woven with nickel-titanium alloy wires, in which the internal space of each disc structure is provided with a polymer occlusion membrane. However, when the typical occluder is used in an interventional operation, the two ends of the occluder tend to be raised after being pulled when the occluder is pulled to withdraw it into a sheath since the occluder has a roughly flat proximal end surface and the alloy wires are converged at the two end surfaces, the occluder cannot be conveniently pulled into the sheath and advanced in the sheath. More importantly, if the occluder is failed to be fastened successfully, the occluder needs to be retracted. At this time, the occluder is located at the rupture in the lesion site and the inner steel cable connected to the occluder is also slightly bent. During retracting, an occlusion portion of the occluder often abuts against a side wall of a distal end port of the sheath, causing problems in the retracting of the occluder, and thus reducing the efficiency of the operation and affecting the success rate of the operation.

### SUMMARY

In view of the shortcomings existing in the prior art, the problem to be solved in the present application is to provide an occluder that is convenient in use and an occluder fastening device having the occluder.

To solve the above technical problems, the present application provides an occluder, which comprises a proximal occlusion portion, wherein the proximal occlusion portion has a proximal end provided with a first converged member, and is provided with a conical guide structure on an outer periphery of the first converged member; and the guide structure has an inner diameter gradually decreasing from a distal end to a proximal end.

The present application also provides an occluder fastening system, which comprises a sheath bendable adjustment device, a loader, a pushing device, and an occluder. The sheath bendable adjustment device includes a sheath. The loader includes a loading tube, wherein a distal end of the loading tube is inserted in the sheath. The pushing device includes a pushing component. The occluder includes a proximal occlusion portion, wherein the proximal occlusion portion has a proximal end provided with a first converged member, and is provided with a conical guide structure on an outer periphery of the first converged member; and the guide structure has an inner diameter gradually decreasing from a distal end to a proximal end. The guide structure provides a guiding performance, to facilitate the pushing component to withdraw the occluder into the sheath.

The proximal occlusion portion of the occluder provided in the present application is provided with the conical guide structure at the proximal end on the outer periphery of the first converged member. When it needs to withdraw the occluder into the sheath, the occluder can easily enter the sheath following the guide structure due to the guiding performance of the guide structure, which is convenient in use.

### BRIEF DESCRIPTION OF THE DRAWINGS

In order to illustrate the technical solutions of the embodiments according to the present application more clearly, drawings used in the description of the embodiments according to the present application will be briefly introduced below. It should be appreciated that the drawings described below merely illustrate some embodiments of the present application, and other variations may be obtained by those skilled in the art without creative effort.

FIG. 1 is a structural schematic view of an occluder according to an embodiment of the present application.

FIG. 2 is a schematic cross-sectional view of FIG. 1, taken along the line II-II.

FIG. 3 is an enlarged view of a connecting bolt shown in FIG. 2.

FIG. 4 is a top view of the connecting bolt shown in FIG. 2.

FIG. 5 is a structural schematic view of an occluder fastening system provided with the occluder shown in FIG. 1.

FIG. 6 is a schematic exploded view of the occluder and a pushing component shown in FIG. 5.

FIG. 7 is a structural schematic view of a distal end surface of a pushing tube of the pushing component shown in FIG. 6.

FIG. 8 is a schematic cross-sectional view of FIG. 6, taken along the line VIII-VIII.

FIG. 9 is a schematic cross-sectional view of FIG. 6, showing a pushing member having been inserted into the pushing tube.

FIG. 10 is a structural schematic view of the pushing device shown in FIG. 5.

FIG. 11 is a schematic exploded view of the pushing device shown in FIG. 10.

FIG. 12 is a schematic cross-sectional view of FIG. 10, taken along the line XII-XII.

FIG. 13 is a structural schematic perspective exploded view of the first shell and the second shell shown in FIG. 11.

FIG. 14 is a structural schematic plan view showing an interior of the first shell shown in FIG. 13.

FIG. 15 is a structural schematic plan view showing an interior of the second shell shown in FIG. 13.

FIG. 16 is a structural schematic plan view showing an external of the second shell shown in FIG. 15.

FIG. 17 is a structural schematic perspective view of an outer buckle plate shown in FIG. 11.

FIG. 18 is a schematic perspective exploded view of an end cap shown in FIG. 11.

FIG. 19 is a cross-sectional view of FIG. 18, taken along the line XIX-XIX.

FIG. 20 is a structural schematic perspective exploded view showing a first clamping block, a movable block, and a second clamping block shown in FIG. 11.

FIG. 21 is a structural schematic view showing one side of the first clamping block shown in FIG. 20.

FIG. 22 is a structural schematic view showing the other side of the first clamping block shown in FIG. 20.

FIG. 23 is a structural schematic side view of the second clamping block shown in FIG. 20.

FIG. 24 is an enlarged view of the movable block shown in FIG. 20.

FIG. 25 is an enlarged view of a button in FIG. 11.

FIG. 26 is a structural schematic view of a first rotating mechanism shown in FIG. 11.

FIG. 27 is a cross-sectional view of FIG. 26, taken along the line XXVII-XXVII.

FIG. 28 is a structural schematic perspective view of a rotating post shown in FIG. 26.

FIG. 29 is a structural schematic perspective view of a second rotating member shown in FIG. 11.

FIG. 30 is a schematic side view of the second rotating member shown in FIG. 29.

FIG. 31 is a cross-sectional view of FIG. 30, taken along the line XXXI-XXXI.

FIG. 32 is an assembled view of the pushing device shown in FIG. 11.

FIG. 33 is a cross-sectional view of FIG. 32, taken along the line XXXI-XXXI.

### DESCRIPTION OF THE EMBODIMENTS

The technical solutions in the embodiments of the present application will be described clearly and fully in conjunction with the accompanying drawings in the embodiments of the present application. Apparently, the embodiments described are merely some, rather than all of the embodiments of the present application. Based on the embodiments of the present application, all other embodiments obtained by a person of ordinary skill in the art without creative efforts shall fall within the protection scope of the present application.

In addition, the following descriptions of various embodiments are provided to exemplify the specific embodiments of the present application with reference to accompanying drawings. The directional terms mentioned in the present application, such as “upper”, “lower”, “front”, “back”, “left”, “right”, “inner”, “outer”, and “side”, etc., are only given with reference to the direction shown in the drawings. Therefore, the directional terms are used to explain more clearly and for better understanding of the present application, without indicating or implying that the device or element referred to requires a specific orientation, a construction and an operation in a specific orientation. Therefore, they cannot be understood as limitations to the present application.

Definitions for the orientations: for clarity of description, in the context with reference to the operation, the end close to the operator is referred to as “proximal end” and the end far away from the operator is referred to as “distal end”. Axial direction refers to a direction parallel to a connection line between a distal center and a proximal center of an instrument. The above definitions are made for convenience of description only and shall not be construed as limit to the present application.

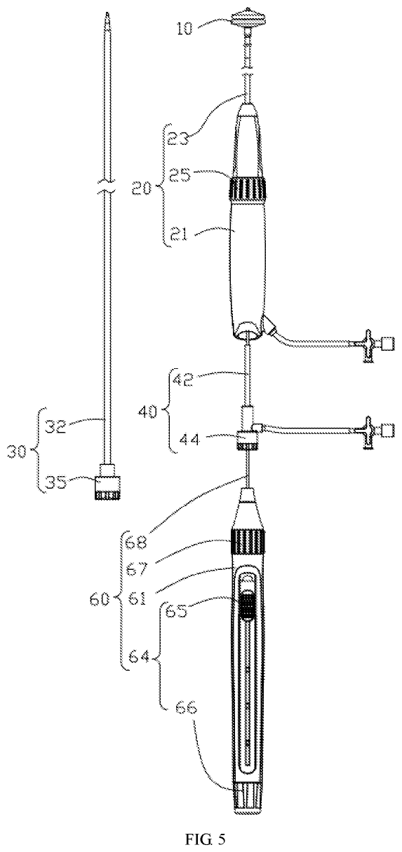

Referring to FIGS. 1 to 4, the present application provides an occluder 10, which includes a proximal occlusion portion 11, a distal occlusion portion 15, and a waist 18 located between the proximal occlusion portion 11 and the distal occlusion portion 15. The proximal occlusion portion 11 has a proximal end provided with a first converged member 110, and a conical guide structure 111 on an outer periphery of the first converged member 110. The guide structure 111 has an inner diameter gradually decreasing from the distal end to the proximal end. In this embodiment, the guide structure 111 is an inverted conical structure.

In the present disclosure, the proximal end of the proximal occlusion portion 11 of the occluder 10 is provided with the conical guide structure 111 on the outer periphery of the first converged member 110, when the occluder 10 is failed to be fastened successfully and required to be retracted into the sheath, the occluder 10 can easily enter the sheath due to the guiding of the guide structure 111, which is convenient in use. The outer diameter of the guide structure 111 at a proximal end thereof is slightly smaller than the inner diameter of a distal end of the sheath, which facilitates the insertion of the proximal end of the guide structure 111 into the sheath when the occluder 10 is retracted.

The first occluder 11, the second occluder 15, and the waist 18 are all woven with metal wires, and form a two-layer disc-shaped structure with an internal space. The middle portions of the metal wires are bent to form the waist 18. The metal wire may be, but is not limited to, nickel-titanium alloy, cobalt-chromium alloy, stainless steel or other metal materials with good biocompatibility, and preferably superelastic shape-memory alloy such as nickel-titanium wire. The proximal occlusion portion 11 and/or the distal occlusion portion 15 is provided with at least one occlusion membrane therein. The occlusion membrane may be fixed in the proximal occlusion portion 11 and the distal end occlusion portion 15 by suturing or bonding. The occlusion membrane may be, but is not limited to, a polytetrafluoroethylene membrane, a polyurethane membrane, or a polyimide membrane.

In other embodiments, the first occluder 11, the second occluder 15, and the waist 18 may be of a grid or frame structure formed by cutting a metal pipe.

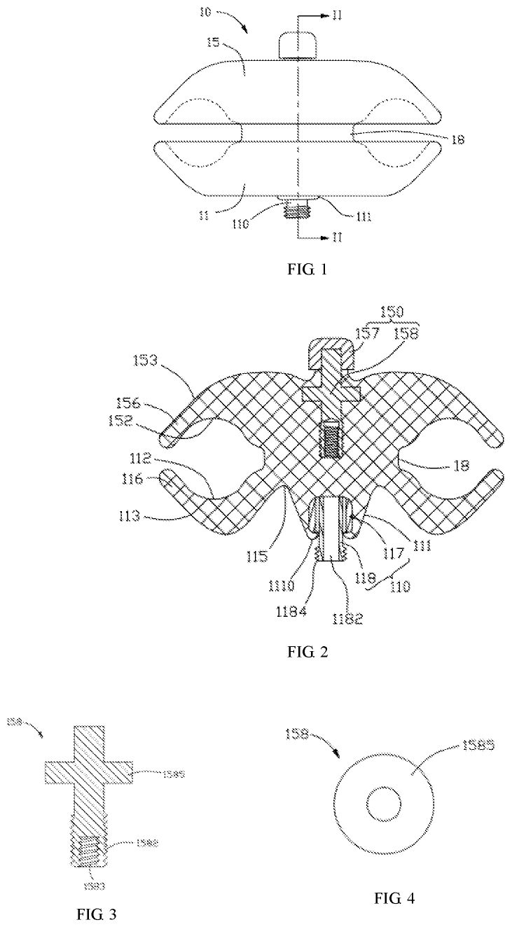

As shown in FIG. 2, the proximal occlusion portion 11 includes a first annular groove 112 extending to the waist 18 and recessed proximally, and a first side wall 113 adjoining an outer edge of the first annular groove 112. The cross section of the first side wall 113 is substantially V-shaped. The first side wall 113 extends obliquely from the outer edge of the first annular groove 112 towards the proximal end for a certain length, and then turns to extend obliquely toward the distal end to be connected to a distal end of the guide structure 111. In this way, the first side wall 113 and the guide structure 111 define an annular valley 115 recessed distally. The first side wall 113 and the first annular groove 112 define an annular first holding shoulder 116, and the cross section of the first holding shoulder 116 is generally V-shaped.

Specifically, The first holding shoulder 116 woven by metal wires is curved and forms an annular recess 112. A radial inner portion of the first holding shoulder 116 is connected to the waist 18. In this embodiment, the first holding shoulder 116 is substantially V shaped in its cross section, having a convex apex protruding proximally and an opening facing distally. In particular, the first holding shoulder 116 has a first section inclinedly extending from the waist 18 towards the proximal end, and a second section inclinedly extending from a tip of the first portion towards the distal end. The second section is inclined with respect to the axial direction of the occlude 10 at an angle ranged from 30° to 45°. When the occluder 10 starts to clamp a rupture, the proximal occlusion portion 11 provides the greatest clamping force, which facilitates the endothelialization of the occluder 10.

The first converged member 110 includes an inner sleeve 117 arranged at the proximal end of the proximal occlusion portion 11 and a protrusion portion 118 fixed to the inner sleeve 117. The proximal ends of the metal wires are converged and positioned between the inner sleeve 117 and the protrusion portion 118. The metal wires between the inner sleeve 117 and the protrusion portion 118 may be fixed by welding, clamping et al.

In this embodiment, the inner sleeve 117 is a steel sleeve with two open ends, and the protrusion portion 118 is a cylinder also having two open ends. The inner diameter of the inner sleeve 117 is larger than the outer diameter of the protrusion portion 118. The inner sleeve 117 may be arranged inside or outside of the proximal occluding portion 11, and the inner sleeve 117 is mounted around the protrusion portion 118. The proximal ends of the metal wires are located between an inner wall of the inner sleeve 117 and an outer wall of the protrusion portion 118. Preferably, the inner sleeve 117 is located inside the proximal occluding portion 11, the distal end of the protrusion portion 118 extends to the interior of the proximal occluding portion 11, the inner sleeve 117 is mounted around the distal end of the protrusion portion 118, and the proximal ends of the metal wires are bent inwardly and then positioned between the inner wall of the inner sleeve 117 and the outer wall of the protrusion portion 118 at the distal end. The inner sleeve 117 is arranged inside the proximal occluding portion 11. The length of the first converged member 110 axially protruding from the axial occluding portion 11 can be reduced after the occluder 10 is fastened, to reduce the risk of postoperative complications. The protrusion portion 118 is axially provided with a through hole 1182, and an external thread 1184 is provided on the outer wall of the protrusion portion 118.

The distal occlusion portion 15 includes a second annular groove 152 extending to the waist 18 and recessed distally, and a second side wall 153 adjoining an outer edge of the second annular groove 152. The second side wall 153 extends obliquely from the second annular groove 152 toward the distal end and converges at a distal end of the distal occlusion portion 15. The second side wall 153 and the second annular groove 152 define a second holding shoulder 156. An outer edge of the second holding shoulder 156 is opposite to the outer edge of the first holding shoulder 116. When the occluder 10 is being used to occlude a rupture, the first holding shoulder 116 and the second holding shoulder 156 clamp two opposite side walls around the rupture.

The distal occlusion portion 15 further includes a second converged member 150 located at the distal end. The metal wires after woven which are adjacent to the second annular groove 152 extend distally towards the second converged member 150 at an angle of 30° to 45° with respect to the axial direction first to form the second side wall 153, and then further approach the second converged member 150 and are fixed to the second converged member 150. When the occluder 10 starts to clamp a rupture, the particular structures of the distal occlusion portion 15 such as the second annular groove 152, the second side wall 153 and the second holding shoulder 156; and of the proximal occlusion portion 11 such as the first annular groove 112, the first side wall 113 and the first holding shoulder 116 provide the maximal clamping force, which facilitates the endothelialization of the occluder 10.

Referring to FIGS. 2 and 3, the second converged member 150 includes an outer sleeve 157 and a connecting bolt 158 connected to a proximal end of the outer sleeve 157. The distal ends of the metal wires are converged and positioned between the outer sleeve 157 and a distal end of the connecting bolt 158. The connecting bolt 158 extends towards the proximal end. An external thread 1582 is provided on an outer wall of the connecting bolt 158 at a proximal end thereof. The outer diameter of the connecting bolt 158 at the proximal end is slightly smaller than the inner diameter of the through hole 1182 of the production portion 118. Moving the connecting bolt 158 towards the proximal end can cause the proximal end of the connecting bolt 158 to extend through the through hole 1182 to be screwed to a nut. The metal wires between the outer sleeve 157 and the connecting bolt 158 can be fixed by welding, clamping et al. One end of the connecting bolt 158 adjacent to the outer sleeve 157 is provided with a protruding plate 1585 in the radial direction. The outer diameter of the protruding plate 1585 is larger than the inner diameter of the through hole 1182 of the protrusion portion 118. The protruding plate 1585 may be circular, square or in other shapes, as long as the protruding plate 1585 can abut against the periphery of the through hole 1182 at the distal end of the protrusion portion 118. The metal wires between the outer sleeve 157 and the connecting bolt 158 can be fixed by welding, clamping et al. Specifically, the outer sleeve 157 can be a cylinder with two open ends and the distal ends of the metal wires are welded to an inner side of the cylinder. The distal open end of the connecting bolt 158 is provided with a protruding surface extending radially, which has a diameter larger than the diameter of the cylinder. The proximal end of the connecting bolt 158 extends through the cylinder and the protruding surface abuts against a distal end surface of the cylinder. In addition, the outer sleeve 157 may have a structure with an inner cavity in a direction from the proximal end to the distal end, wherein the distal end of the connecting bolt 158 is fixed in the inner cavity and the metal wires are welded between the external sleeve 157 and the connecting bolt 158. In this embodiment, the latter structure is employed.

If there is no protruding plate 1585 provided on the connecting bolt 158, during the fastening process of the occluder 10, the connecting bolt 158 is pulled toward the proximal end, the second occluder 15 woven by metal wires which is fixed to the connecting bolt 158 may be pulled into the rupture through the through hole 1182 of the protrusion portion 118 when the force applied to the connecting bolt 158 is too large, causing a circumference of the second occluder 15 to warp to form a flared configuration. The warped second occluder 15 cannot provide a clamping force on the tissue around the rupture, which affects the occlusion performance on the rupture. However, in the occluder 10 according to the present application, when the connecting bolt 158 pulls the distal occlusion portion 15 to approach the rupture, the protruding plate 1585 on the connecting bolt 158 can abut against the distal end of the protrusion portion 118, to provide a stop performance. This can ensure that the distal occlusion portion 15 is located in the cavity of the occluder 10 without being pulled into the rupture by the connecting bolt 158, thus preventing the circumference of the second occluder 15 from warping and ensuring that the first holding shoulder 116 and the second holding shoulder 156 have a good clamping performance for the rupture and the occlusion membrane inside the proximal occlusion portion 11, the distal occlusion portion 15 and the waist 18.

Before the connecting bolt 158 is fastened to the nut, the connecting bolt 158 is located inside the occluder 10, and the proximal end of the connecting bolt 158 extends through the waist 18 of the occluder 10. A screw hole 1583 is provided in a central portion of a proximal end surface of the connecting bolt 158 in the axial direction.

The first annular groove 112 and the second annular groove 152 are arranged oppositely at the proximal end and the distal end of the waist 18. The first annular groove 112 and the second annular groove 152 cooperatively define an annular space. When the occluder 10 is mounted to the rupture, the tissue around the rupture is inserted into the annular space. The first holding shoulder 116 and the second holding shoulder 156 can hold the two opposite side walls of the tissue around the rupture, and further facilitates the endothelialization of the occluder.

Preferably, the threading direction of the external thread 1582 of the connecting bolt 158 is opposite to the threading direction of the external thread 1184 of the protrusion portion 118. Specifically, when the external thread 1582 of the connecting bolt 158 is a left-hand thread, then the external thread 1184 of the protrusion portion 118 is a right-hand thread; and when the external thread 1582 of the connecting bolt 158 is a right-hand thread, then the external thread 1184 of the protrusion portion 118 is a left-hand thread.

In other embodiments, the occluder 10 may not be provided with the inner sleeve 117 and the outer sleeve 157. The distal ends of the metal wires are converged and positioned at the distal end of the connecting bolt 158 by welding, and the proximal ends of the metal wires are converged and positioned at the distal end of the protrusion portion 118 by welding.

Referring to FIG. 5. the present application also provides an occluder fastening system, which includes an occluder 10, a sheath bendable adjustment device 20, an expander 30, a loading component 40 disposed at a proximal end of the sheath bendable adjustment device 20, and a pushing device 60 disposed at a proximal end of the loading component 40. The sheath bendable adjustment device 20 includes a casing 21, a sheath 23 having a proximal end received in the casing 21, and an adjustment means 25 for bending a bendable distal end of the sheath 23. The loading component 40 includes a loading tube 42 and a sealing seat 44 provided at a proximal end of the loading tube 42. A distal end of the loading tube 42 can be inserted in the sheath 23. The pushing device 60 can push the occluder 10. The pushing device 60 includes a housing 61 with a receiving space, a first pushing component 64 partially received in the housing 61, a second pushing component 67 partially mounted around the housing 61, and a pushing component 68. A proximal end of the pushing component 68 is received in the housing 61.

The expander 30 has an elongated cylindrical structure. The diameter of the expander 30 at the distal end thereof is gradually reduced to form a cone-like structure. Specifically, the expander 30 includes an expansion rod 32 and a connecting portion 35 provided at a proximal end of the expansion rod 32. A distal end of the expansion rod 32 gradually decreases in diameter to form a cone-like structure. The cone-like structure facilitates the insertion of the expansion rod 32 into the sheath 23. During the operation, it needs to install the expander 30 into the sheath bendable adjustment device 20 to form an expansion assembly.

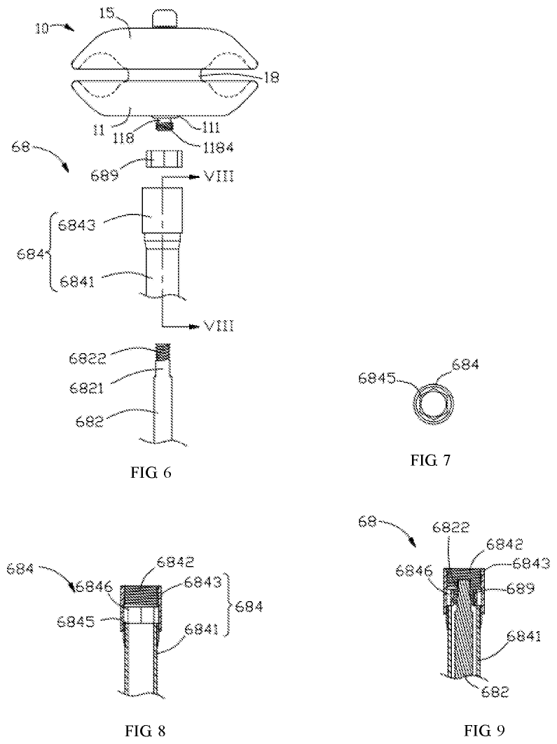

As shown in FIGS. 5 to 9, the pushing component 68 includes a pushing member 682, a pushing tube 684 movably mounted around the pushing member 682, and a nut 689 positioned inside a distal end of the pushing tube 684. A distal end of the pushing member 682 is detachably connected to the connecting bolt 158 of the occluder 10. An inner wall of the pushing tube 684 at a distal end thereof is provided with an internal thread 6842 corresponding to the external thread 1184 of the protrusion portion 118. The first pushing component 64 includes a sliding means 65 which can drive the pushing member 682 to axially slide back and forth in the pushing tube 684, to drive the connecting bolt 158 to abut against or move away from the screw hole of the nut 689. The second pushing component 67 includes a rotating means that can drive the pushing tube 684 to rotate, and thus the nut 689 is driven to rotate so that the connecting bolt 158 is screwed to the nut 689. During the process of screwing the connecting bolt 158 into the nut 689, since the threading direction of the external thread 1582 of the connecting bolt 158 is opposite to the threading direction of the external thread 1184 of the protrusion portion 118, that means, the screwing direction between the connecting bolt 158 and the nut 689 is opposite to the screwing direction between the external thread 1184 of the protrusion portion 118 and the internal thread 6842 of the pushing tube 684, the external thread 1184 of the protrusion portion 118 can be disengaged from the internal thread 6842 of the pushing tube 684. In other words, when the connecting bolt 158 is screwed to the nut 689, the disengagement of the protrusion portion 118 from the pushing tube 684 can be achieved in the meanwhile. When the connecting bolt 158 is disengaged from the nut 689, the threaded connection between the protrusion portion 118 and the pushing tube 684 is achieved in the meanwhile. Therefore, the occlude 10 not only has a high reliability with good occluding performance, but is also convenient and simple to operate.

Specifically, the pushing component 68 is a steel cable assembly, in which the pushing member 682 is a steel cable with good elasticity, and the pushing tube 684 is a steel tube with good elasticity. The steel cable is movably inserted in the steel duct. The pushing tube 684 includes a body section 6841 and an extension section 6843 disposed at a distal end of the body section 6841. The nut 689 can be positioned in the extension section 6843. The internal thread 6842 is located on an inner wall of the extension section 6843 at a distal end thereof. The internal thread 6842 is engageable with the external thread 1184 of the protrusion portion 118. When the occluder 10 is being connected with the pushing component 68, the screwing direction between the extension section 6843 and the protrusion portion 118 is opposite to the screwing direction between the connecting bolt 158 and the nut 689. The threads of the extension section 6843 and the protrusion portion 118 have a number of turns and thread pitch equaling to or greater than those of the thread of the nut 689 so that the proximal end of the connecting bolt 158 extends out of a proximal end surface of the nut 689.

The outer wall of the pushing member 682 at the distal end thereof is provided with an external thread 6822 corresponding to the screw hole 1583 of the connecting bolt 158. The distal end of the pushing member 682 can movably extend through the nut 689 and the extension section 6843. Specifically, A distal end surface of the pushing member 682 is provided with a cylindrical connecting rod 6821 at a central part thereof which protrudes axially therefrom. The outer diameter of the connecting rod 6821 is smaller than the outer diameter of a proximal portion of the pushing member 682. The external thread 6822 is provided on an outer wall of the connecting rod 6821 at a distal end thereof.

The outer diameter of the body section 6841 is smaller than the outer diameter of the extension section 6843, the inner diameter of the body section 6841 is smaller than the inner diameter of the extension section 6843, such that a step surface 6845 is formed in the pushing tube 684 between a proximal end of the extension section 6843 and a distal end of the body section 6841, against which the nut 689 can abut. An inner wall at a proximal end of the extension section 6843 is provided with an engaging groove 6846. The nut 689 can be engaged in the engaging groove 6846. The engaging groove 6846 can prevent the nut 689 from rotating in the pushing tube 684. The nut 689 can axially slide in the extension section 6843. Specifically, the engaging groove 6846 is formed by being recessed radially inwardly and regularly from the inner wall of the extension section 6843 at the proximal end. The engaging groove 6846 can be engaged with the nut 689. The nut 689 can be, for example, a hexagonal nut. The engaging groove 6846 is a hexagonal groove corresponding to the hexagonal nut. That is, the radial cross section of the engaging groove 6846 is hexagonal. Therefore, when the nut 689 is engaged in the engaging groove 6846, the nut 689 cannot rotate relative to the pushing tube 684, but the nut 689 axially slides in the extension section 6843, and the nut 689 abuts against the step surface 6845.

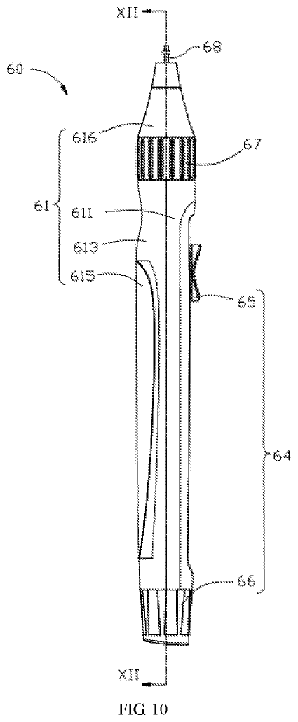

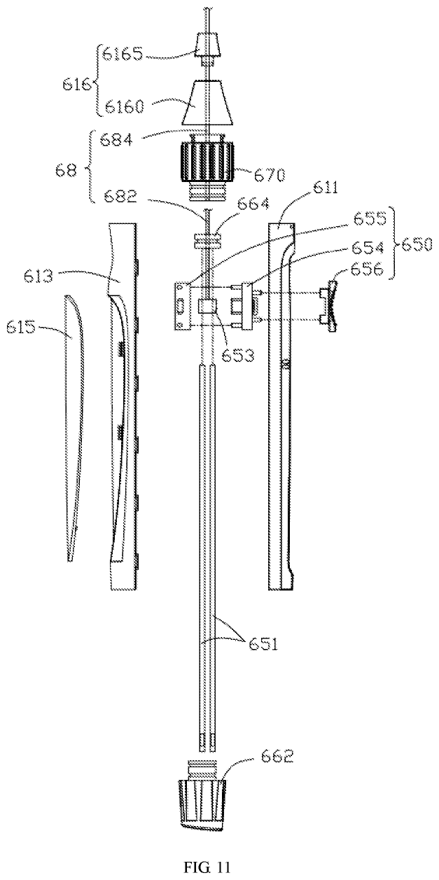

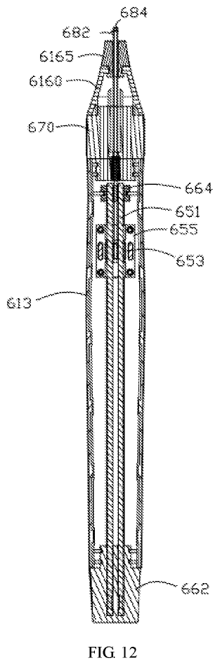

Referring to FIGS. 10 to 12, the first control component 64 includes a sliding means 65 and a first rotating means 66. The sliding means 65 can drive the pushing member 682 to slide axially and be positioned in the pushing tube 684. The first rotating means 66 can drive the pushing member 682 to rotate in the pushing tube 684. The second control component 67 includes a second rotating means 670. The second rotating means 670 can drive the pushing tube 684 to rotate outside the pushing member 682.

The sliding mechanism 65 of the pushing device 60 can drive the pushing member 682 to slide axially in the pushing tube 684, which in turn drives the proximal end of the connecting bolt 158 to extend through the through hole 153 of the protrusion portion 118 to abut against or move away from the nut 689 positioned in the pushing tube 684. The first rotating mechanism 66 can drive the pushing member 682 to rotate in the pushing tube 684, to achieve the threaded connection or disengagement of the external thread 6822 of the pushing member 682 and the screw hole 1583 of the connecting bolt 158. The second rotating mechanism 670 can drive the pushing tube 684 to rotate outside the pushing member 682, to drive the nut 689 to rotate, so that the nut 689 is screwed to the connecting bolt 158 and the external thread 1184 of the protrusion portion 118 is disengaged from the internal thread 6842 of the pushing tube 684; or the nut 689 is disengaged from the connecting bolt 158 and the external thread 1184 of the protrusion portion 118 is screwed to the internal thread 6842 of the pushing tube 684. The pushing device 60 is simple to operate and convenient in use, which improves the reliability and efficiency of the pushing device 60, thereby reducing the operation time.

The housing 61 includes a first shell 611, a second shell 613, an outer buckle plate 615 and an end cover 616. The first shell 611 is fitted to the second shell 613 to form a tubular structure with two open ends. The sliding means 65 is axially arranged in the tubular structure. The first rotating means 66 is arranged at a proximal end of the tubular structure. The second rotating means 670 is arranged at a distal end of the tubular structure. The end cover 616 is arranged at a distal end of the second rotating means 670.

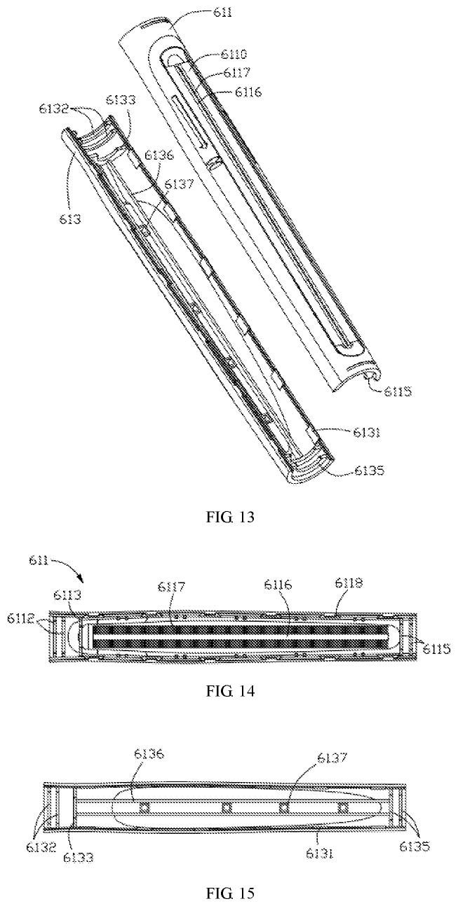

As shown in FIGS. 13 and 15, an inner wall of the first shell 611 is provided with at least one first support sheet 6112 and at least one second support sheet 6113 at a distal end thereof along the axial direction. The inner wall of the first shell 611 is provided with at least one third support sheet 6115 at a proximal end thereof. The first support sheet 6112, the second support sheet 6113 and the third support sheet 6115 are all semi-circular ring-shaped sheets. The first support sheet 6112 and the second support sheet 6113 are arranged at an interval. The axes of the first support sheet 6112, the second support sheet 6113 and the third support sheet 6115 all extend along the axial direction, and the axes of the first support sheet 6112, the second support sheet 6113 and the third support sheet 6115 coincide each other.

In this embodiment, two first support sheets 6112 are provided, and the two first support sheets 6112 are spaced apart axially and parallel with each other. One second support sheet 6113 is provided. Two third support sheets 6115 are provided, and the two third support sheets 6115 are spaced apart axially and parallel with each other.

In other embodiments, other numbers of the first support sheet 6112, the second support sheet 6113, and the third support sheet 6115 may be provided.

The outer wall of the first shell 611 is configured to have a rectangular plane 6110, and the length of the plane 6110 extends along the axial direction, that is, the plane 6110 extends from the distal end to the proximal end. The plane 6110 is provided with a strip-shaped opening 6116 along the axial direction. The strip-shaped opening 6116 is located between the second support sheet 6113 and the third support sheet 6115. The strip-shaped opening 6116 extends from the distal end to the proximal end of the first shell 611. The inner wall of the first shell 611 is provided with a rack 6117 on at least one side of the strip-shaped opening 6116, and the rack 6117 extends from the distal end to the proximal end of the strip-shaped opening 6116. Two opposite side walls of the first shell 611 are respectively provided with a plurality of engaging pieces 6118 protruding there from.

In this embodiment, the inner wall of the first shell 611 is provided with two strip-shaped grooves along the axial direction on opposite sides of the strip-shaped opening 6116, and each strip-shaped groove is provided with a rack 6117.

As shown in FIGS. 13, 15 and 16, the structure of the second shell 613 is similar to the structure of the first shell 611. An inner wall of the second shell 613 is provided with at least one fourth support sheet 6132 and at least one fifth support sheet 6133 at a distal end thereof along the axial direction. The inner wall of the second shell 613 is provided with at least one sixth support sheet 6135 at a proximal end thereof. The fourth support sheet 6132, the fifth support sheet 6133 and the sixth support sheet 6135 are all semi-circular ring-shaped sheets. The fourth support sheet 6132 and the fifth support sheet 6133 are arranged at an interval. The axes of the fourth support sheet 6132, the fifth support sheet 6133 and the sixth support sheet 6135 all extend along the axial direction, and the axes of the fourth support sheet 6132, the fifth support sheet 6133 and the sixth support sheet 6135 coincide. When the first shell 611 is fitted to the second shell 613, the first support sheet 6112, the second support sheet 6113 and the third support sheet 6115 of the first shell 611 are respectively corresponding to the fourth support sheet 6132, the fifth support sheet 6133 and the sixth support sheet 6135 of the second shell 613. The first support sheet 6112 and the corresponding fourth support sheet 6132 cooperatively define a ring-shaped support sheet, the second support sheet 6113 and the fifth support sheet 6133 cooperatively define a ring-shaped support sheet, and the third support sheet 6115 and the corresponding sixth support sheet 6135 cooperatively define a ring-shaped support sheet.

In this embodiment, two fourth support sheets 6132 are provided, and the two fourth support sheets 6132 are spaced apart and parallel with each other. One fifth support sheet 6133 is provided. Two sixth support sheets 6135 are provided, and the two sixth support sheets 6135 are spaced apart and parallel with each other.

In other embodiments, other numbers of the fourth support sheet 6132, the fifth support sheet 6133, and the sixth support sheet 6135 may be provided.

At least one rail 6136 protrudes from the inner wall of the second shell 613 along the axial direction. The rail 6136 is located between the fifth support sheet 6133 and the sixth support sheet rail 6135, and the rail 6136 extends from the distal end to the proximal end. In this embodiment, two rails 6136 spaced apart and parallel with each other protrude from the middle portion of the inner wall of the second shell 613, and a plurality of positioning blocks 6137 are axially arranged at intervals between the two rails 6136. Each positioning block 6137 is provided with an engaging hole. The outer wall of the second shell 613 is provided with a mounting opening 6138 at a central part thereof, and the second shell 613 is provided with a plurality of engaging holes 6139 around the mounting opening 6138. Two opposite side walls of the second shell 613 are respectively provided with a plurality of elastic engaging hooks 6131 protruding therefrom, which are corresponding to the engaging pieces 6118 of the first shell 611 in a one-to-one manner.

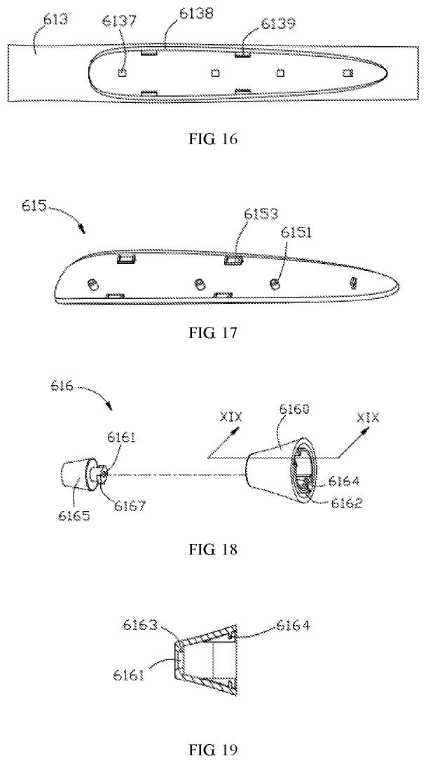

As shown in FIGS. 16 and 17, the outer buckle plate 615 is a convex arc-shaped sheet. The outer buckle plate 615 is corresponding to the mounting opening 6138 of the second shell 613. A side of the outer buckle plate 615 facing the mounting opening 6138 is provided with a plurality of positioning posts 6151 and a plurality of engaging hooks 6153 protruding therefrom. These positioning posts 6151 are corresponding to the engaging holes in the positioning blocks 6137 of the second shell 613 in a one-to-one manner. These engaging hooks 6153 are corresponding to the engaging holes 6139 of the second shell 613 in a one-to-one manner. The outer wall of the outer buckle plate 615 is provided with anti-slip strips or bulges to increase the friction, for facilitating the grip of the pushing device 60 by fingers and facilitating the operation of the pushing device 60.

As shown in FIGS. 18 and 19, the end cover 616 has a conical structure. The end cover 616 includes a cover body 6160 and a slipcover 6165 connected to a distal end of the cover body 6160. The end cover 616 is axially provided with a through hole 6161, and the through hole 6161 extends through a central portion of the cover body 6160 and the slipcover 6165. A tapered cavity is defined inside the cover body 6160, and an annular groove 6162 is provided on an inner wall of the cover body 6160 at the proximal end. The axis of the annular groove 6162 coincides with the axis of the through hole 6161. Two opposing engaging blocks 6164 are arranged in the tapered cavity at the proximal end of the cover body 6160.

The slipcover 6165 is engaged to the distal end of the cover body 6160. Specifically, an engaging ring protrudes from an inner peripheral surface of the through hole 6161 at the distal end of the cover body 6160, and an extension post surrounding the through hole 6161 protrudes from a proximal end surface of the slipcover 6165. A proximal end of the extension post is radially provided with a pair of protruding sheets. After the slipcover 6165 is inserted into the through hole 6161 of the cover body 6160 by means of the extension tube, the protruding sheet 6167 is engaged to the engaging ring 6163 of the cover body 6160, to fix the slipcover 6165 to the cover body 6160. The slipcover 6165 is made of soft materials such as rubber and silicone. The proximal end of the pushing component 68 extends through the through hole 6161 of the slipcover 6165 and the cover body 6160 and is then connected into the housing 61. Since the slipcover 6165 is made of a soft material, the slipcover 6165 can reduce the friction and wear of the pushing component 68 with the distal end port of the end cover 616 caused by the wobbling.

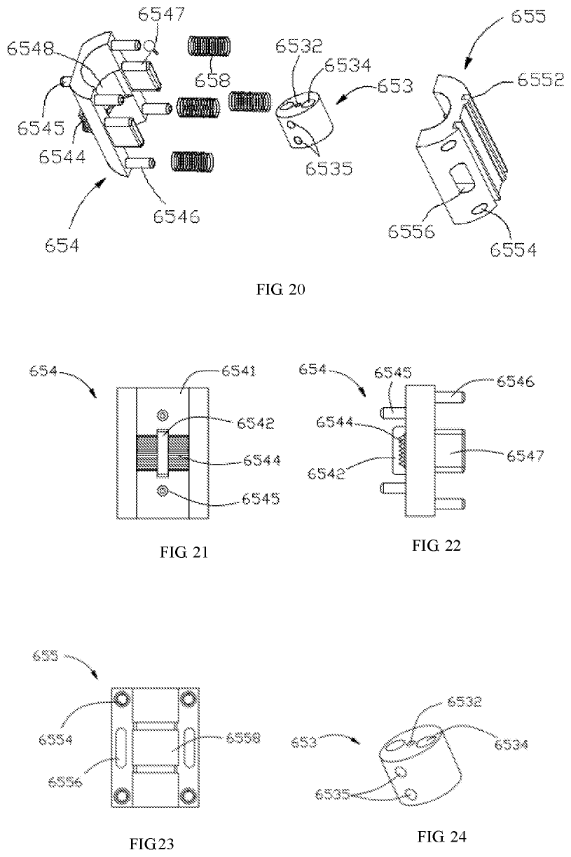

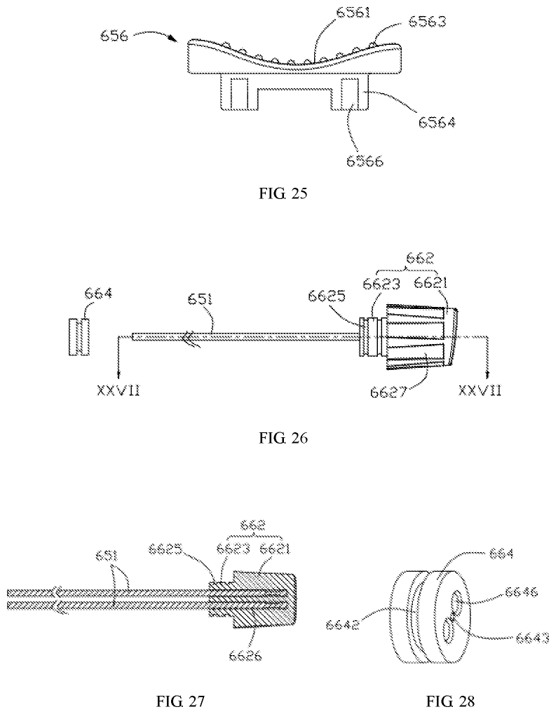

Referring to FIGS. 2 to 31, the sliding means 65 is slidably disposed in the housing 61 along the axial direction. The sliding means 65 includes at least one guide rail 651 that is disposed to extend in the housing 61 along the axial direction, a movable block 653 that is slidably arranged around the at least one guide rail 651, and a sliding member 650 that can drive the movable block 653 to slide along the guide rail 651. A proximal end of the pushing member 682 is fixed to the movable block 653. The sliding member 650 includes a first clamping block 654, a second clamping block 655, and a button 656 partially exposed from the housing 61 and connected to the sliding member 650. The first clamping block 654 can be moved close to or away from the second clamping block 655. The movable block 653 is arranged between the first clamping block 654 and the second clamping block 655. The first clamping block 654 and the second clamping block 655 can be moved close to each other to tightly clamp the movable block 653 therebetween. The first clamping block 654 and the second clamping block 655 can also be moved away from each other to release the movable block 653. The movable block 653 can rotate.

In this embodiment, two guide rails 651 are provided in the housing 61 which are axially arranged, spaced apart and parallel with each other.

The first clamping block 654 has a semi-circular tube structure. A rectangular top surface 6541 is provided in a central part of an outer wall of the first clamping block 654. The length direction of the top surface 6541 extends along the axial direction of the first clamping block 654. Partial structure of the first clamping block 654 can extend through the strip-shaped opening 6116 and slide there along. Specifically, a sliding strip 6542 protrudes from a central part of the top surface 6541, and the length direction of the sliding strip 6542 extends along the length direction of the top surface 6541. The sliding strip 6542 can be inserted into the strip-shaped opening 6116 of the first shell 611 and is slidable therein. The top surface 6541 is provided with engaging teeth 6544 on at least one side of the sliding strip 6542. The engaging teeth 6544 can be engaged to the corresponding rack 6117 of the first shell 611. Preferably, the top surface 6541 is provided with engaging teeth 6544 on two opposite sides of the sliding strip 6542, and the engaging teeth 6544 on two opposite sides can be respectively engaged to the two racks 6117 of the first shell 611. Two connecting posts 6545 respectively protrudes from the two opposite ends of the sliding strip 6542 on the top surface 6541. Two sides of the first clamping block 654 facing away from the top surface 6541 are each provided with two guide posts 6546 and a guide plate 6547 protruding therefrom. On each side, the guide plate 6547 is located between the two guide posts 6546. The first clamping block 654 is provided with a first clamping groove 6548 on an inner wall thereof at a central part, and the first clamping groove 6548 can receive the outer wall of the movable block 653.

The second clamping block 655 has a semi-circular tube structure. Two guide grooves 6552 spaced apart from each other are axially provided in a central part of an outer wall of the second clamping block 655. The two guide grooves 6552 correspond to the rails 6136 of the second shell 613, and the second clamping block 655 is slidable along the rails 6136. Two opposite sides of the second clamping block 655 each are provided with two guide holes 6554 and an access hole 6556. On each side, the access hole 6556 is located between the two guide holes 6554. A second clamping groove 6558 is provided in a central part of an inner wall of the second clamping block 655, and the second clamping groove 6558 can receive the outer wall of the movable block 653.

The movable block 653 is a cylindrical structure. The proximal end of the pushing member 682 is fixed to a central part of the movable block 653. Specifically, a central part of a distal end surface of the movable block 653 is provided with a fixing hole 6532 along the axial direction, and the proximal end of the pushing member 682 is fixed in the fixing hole 6532. The end surface of the movable block 653 is provided with two through holes 6534 at two opposite sides of the fixing hole 6532. Each through hole 6534 extends in the axial direction and penetrates through the proximal end surface and the distal end surface of the movable block 653. Each through hole 6534 is configured for one guide rail 651 extending therein. The outer wall of the movable block 653 is provided with a plurality of welding holes 6535 communicating with the fixing hole 6532, and the pushing member 682 is fixed to the movable block 653 by solder added into the welding hole 6535.

An elastic member 658 is provided between the first clamping block 654 and the second clamping block 655, and the elastic member 658 can force the first clamping block 654 to move away from the second clamping block 655. Preferably, the elastic member 658 is a spring mounted outside the guide post 6546.

A recess 6561 is provided in a central part of the side wall of the button 656, and a number of anti-slip strips 6563 are arranged on the surface of the recess 6561. A finger can be inserted into the recess 6561 to facilitate the operation of the button 656. Two connecting blocks 6564 respectively protrudes from a side of the button 656 facing away from the recess 6561 at two opposite ends. Each connecting block 6564 is provided with a connecting hole 6566 on a side facing away from the recess 6561. The two connecting holes 6566 are corresponding to the two connecting posts 6545 of the first clamping block 654.

In other embodiments, the button 656 may be integrally formed with the first clamping block 654, that is, the first clamping block 654 is provided with a button extending through the strip-shaped opening 6116.

FIGS. 26 to 28 are structural schematic views showing the first rotating mean 66. The first rotating means 66 includes a first rotating member 662 fixed to the guide rail 651, and a rotating post 664 arranged in the housing 61 which is rotatable about the axis of the pushing member 682. Specifically, the first rotating means 66 is fixedly connected to the proximal end of the guide rail 651, and the rotating post 664 is fixedly connected to the distal end of the guide rail 651. The axis of the guide rail 651 is spaced apart from and parallel to the axis of the pushing member 682. The first rotating member 662 is arranged at the proximal end of the housing 61 rotatable about the axis of the pushing member 682. The rotation of the first rotating member 662 can drive the movable block 653 and the rotating post 664 to rotate through the guide rail 651, thereby causing the pushing member 682 to rotate.

Specifically, the first rotating member 662 includes a handle portion 6621 and a rotating rod 6623 protruding from a central part of a distal end surface of the handle portion 6621. The rotating rod 6623 is a cylinder. An outer wall of the rotating rod 6623 is provided with two annular guide grooves 6625 along its circumferential direction. The two guide grooves 6625 correspond to the ring-shaped support sheets defined by the third support sheets 6115 of the first shell 611 and the sixth support sheets 6135 of the second shell 613. The axes of the two guide grooves 6625 coincide with the axis of the first rotating member 662. The first rotating member 662 can rotate along the guide groove 6625. An end surface of the rotating rod 6623 facing away from the handle portion 6621 is provided with two spaced fixing holes 6626. The axes of the two fixing holes 6626 do not coincide with the axis of the rotating rod 6623. The proximal ends of the two guide rail 651 are fixed respectively in the two fixing holes 6626. A number of anti-slip grooves 6627 are provided on the outer wall of the handle portion 6621 to facilitate the rotation of the first rotating member 662.

The rotating post 664 is a cylinder. An outer wall of the rotating post 664 is provided with a guide groove 6642 along its circumference. The guide groove 6642 corresponds to the ring-shaped support sheet defined by the second support sheet 6113 of the first shell 611 and the fifth support sheet 6133 of the second shell 613. The rotating post 664 can rotate along the guide groove 6642. A central part of the rotating post 664 is axially provided with a through hole 6643 extending through a proximal end surface and a distal end surface thereof. The pushing member 682 can movably extend through the through hole 6643. The axis of the through hole 6643 coincides with the axis of the guide groove 6642. The proximal end surface of the rotating post 664 has two fixing holes 6646 provided on two opposite sides of the through hole 6643, and the distal ends of the two guide rails 651 are respectively fixed in the two fixing holes 6646.

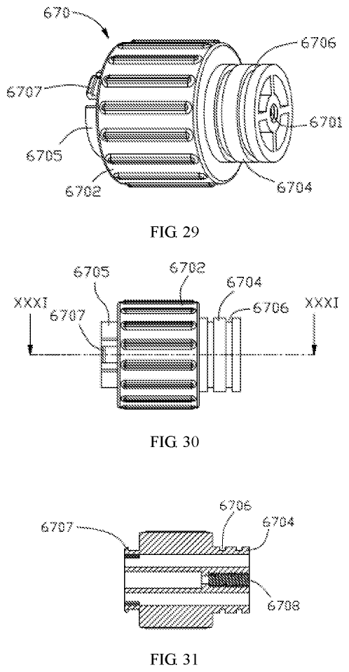

FIGS. 29 and 30 are structural schematic views showing the second rotation member 670, The second rotating member 670 is arranged at the distal end of the housing 61 rotatable about the axis of the pushing member 682. The second rotating member 670 is a cylinder. A central part of the second rotating member 670 is axially provided with a through hole 6701, and the proximal end of the pushing tube 684 is fixed in the through hole 6701. The pushing member 682 movably extends through the through hole 6701. The axis of the through hole 6701 coincides with the axis of the pushing tube 684, and the axis of the second rotating member 670 coincides with the axis of the through hole 6701.

Specifically, the second rotating member 670 includes a cylindrical operating portion 6702, a rotating portion 6704 protruding from a central part of a proximal end surface of the operating portion 6702, and an annular protruding sheet 6705 protruding from a distal end surface of the operating portion 6702. An outer wall of the operating portion 6702 is provided with anti-skip strips, which facilitate the rotation of the second rotating member 670 by fingers. The rotating portion 6704 is a cylinder. An outer wall of the rotating portion 6704 is provided with two annular guide groove 6706 along its circumference. The two guide grooves 6706 correspond to the annular support sheets defined by the first support sheets 6112 of the first shell 611 and the fourth support sheets 6132 of the second shell 613. The axes of the two guide grooves 6706 coincide with the axis of the through hole 6701, and the second rotating member 670 can rotate along the guide groove 6706. The axis of the annular protruding sheet 6705 coincides with the axis of the through hole 6701. Two opposite engaging hooks 6707 are provided on the annular protruding sheet 6705 in the radial direction, and the two engaging hooks 6707 correspond to the two engaging blocks 6164 of the cover body 6160. An internal thread 6708 is provided on an inner wall of the through hole 6701 at the proximal end, and an external thread is provided on an outer wall of the pushing tube 684 at the proximal end corresponding to the internal thread 6708.

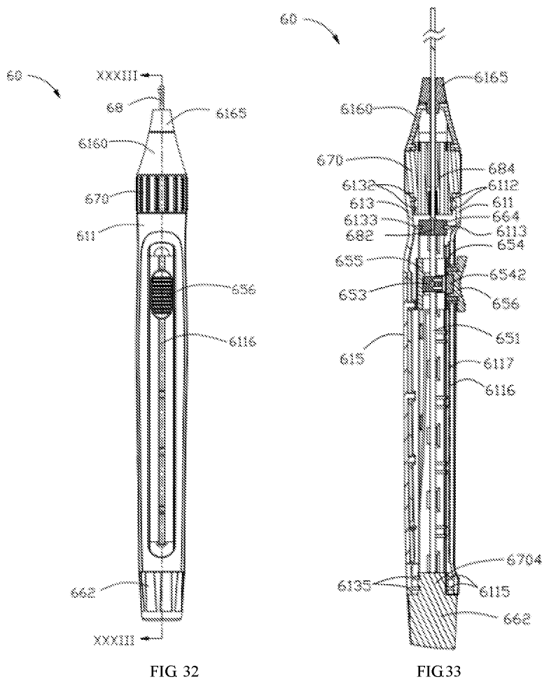

Referring also to FIGS. 10 to 33, when assembling the pushing device 60, the proximal end of the pushing member 682 movably extends through the through hole 6643 of the rotating post 664 and then is fixed in the fixing hole 6532 of the movable block 653. The two guide rails 651 slidably extend through the two through holes 6534 of the movable block 653 respectively, with the distal ends of the two guide rails 651 being fixed in the two fixing holes 6646 of the rotating post 664 respectively and the proximal ends of the two guide rails 651 being fixed in the two fixing holes 6626 of the first rotating member 662. The movable block 653 is slidably mounted around the guide rails 651 and located between the first rotating member 662 and the rotating post 664. The elastic members 658 are respectively mounted around the guide posts 6546. The movable block 653 is arranged between the first clamping block 654 and the second clamping block 655. The first clamping block 654 is fitted to the second clamping block 655, such that the movable block 653 is movably clamped in a receiving space defined by the first clamping groove 6548 of the first clamping block 654 and the second clamping groove 6558 of the second clamping block 655. The guide posts 6546 are slidably inserted into the corresponding guide holes 6554, and the elastic members 658 are elastically pressed between first clamping block 654 and second clamping block 655. The first clamping block 654 is moved close to the second clamping block 655 so that the guide plate 6547 extends into and is engaged in the access hole 6556, and is able to drive the second clamping block 655 to slide. The outer wall of the guide plate 6547 abuts against the inner peripheral surface of the access hole 6556 without a gap between the guide plate 6547 and the access hole 6556, thereby preventing the first clamping block 655 from moving relative to the second clamping block 655 in the axial direction. As such, the movable block 653 can be driven to and positioned at a proper position more conveniently and accurately. The two guide rails 651 are located between the first shell 611 and the second shell 613 in such a manner that the connecting posts 6545 of the first clamping block 654 is opposite to the strip-shaped opening 6116 of the first shell 611. The second support sheet 6113 of the first shell 611 is inserted into the guide groove 6642 of the rotating post 664, and the two third support sheets 6115 are respectively inserted into the two guide grooves 6625 of the first rotating member 662. At this time, the sliding strip 6542 and the connecting post 6545 are both inserted into the strip-shaped opening 6116, the sliding strip 6542 can slide axially along the strip-shaped opening 6116, and the two sets of engaging teeth 6544 of the first clamping block 654 correspond to two racks 6117 respectively. The two connecting blocks 6564 of the button 656 are mounted around the two connecting posts 6545, and the recess 6561 of the button 656 exposes from the first shell 611. The button 656 can slide along the strip-shaped opening 6116.

The slipcover 6165 is fitted to the distal end of the cover body 6160. Specifically, the pair of protruding sheets 6167 of the slipcover 6165 are engaged to the engaging ring 6163 of the cover body 6160. The end cover 616 is engaged to the distal end of the second rotating member 670. Specifically, the annular protruding sheet 6705 is inserted into the annular groove 6162 of the cover body 6160 and the two engaging hooks 6707 are respectively engaged to the two engaging blocks 6164, such that the end cover 616 is fixed to the second rotating member 670. The axis of the through hole 6161 of the end cover 616 coincides with the axis of the through hole 6701 of the second rotating member 670. In other embodiments, the engaging hooks 6707 of the second rotating member 670 and the engaging blocks 6164 of the cover body 6160 can be omitted, and the annular protruding sheet 6705 of the second rotating member 670 is rotatably inserted into the annular groove 6162 of the cover body 6160, so that the end cover 616 is rotatably connected to the distal end of the second rotating member 670. The proximal end of the pushing tube 684 is caused to extend through the through hole 6161 of the end cover 616 and then fixed in the through hole 6701 of the second rotating member 670. Specifically, the external thread of the pushing tube 684 is screwed to the internal thread 6708 of the second rotating member 670. In other embodiments, the proximal end of the pushing tube 684 can be fixed in the through hole 6161 by welding, gluing or snap-fitting.

The distal end of the pushing member 682 is caused to extend through the through hole 6701 and the pushing tube 684 from the proximal end of the second rotating member 670, such that the second rotating member 670 is caused to slide to the distal end of the first shell 611. The two first support sheets 6112 of the first shell 611 are separately inserted into the two guide grooves 6706 of the second rotating member 670. The second shell 613 is connected to the first shell 611, and the engaging hooks 6131 of the second shell 613 are respectively engaged to the engaging pieces 6118 of the first shell 611. At this time, the two fourth support sheets 6132 of the second shell 613 are respectively inserted into the two guide grooves 6706 of the second rotating member 670, the fifth support sheet 6133 is received in the guide groove 6642 of the rotating post 664, the two sixth support sheets 6135 are respectively inserted into the two guide grooves 6625 of the first rotating member 662, the two rails 6136 are respectively received in the two guide grooves 6552 of the second clamping block 655, the elastic member 658 pushes the first clamping block 654 to cause the engaging teeth 6544 to be engaged to the racks 6117, and the operating portion 6702 of the second rotating member 670 is exposed from the housing 61. Then the outer buckle plate 615 is engaged into the mounting opening 6138 of the second shell 613. Specifically, the positioning posts 6151 of the outer buckle plate 615 are engaged into the engaging holes of the respective positioning blocks 6137, and the engaging hooks 6153 are engaged to the corresponding engaging holes 6139 such that the installation is completed.

When using the pushing device, the button 656 is pressed to move the first clamping block 654 towards the second clamping block 655. The elastic members 658 are resiliently deformed until the movable block 653 is clamped and positioned by the first clamping block 654 and the second clamping block 655. The engaging teeth 6544 of the first clamping block 654 are disconnected from the racks 6117. The button 656 is caused to slide towards the proximal end or the distal end along the strip-shaped opening 6116 of the first shell 611, so that the movable block 653 slides proximally or distally along with the first clamping block 654 and the second clamping block 655 to drive the pushing member 682 to slide proximally or distally in the pushing tube 684, thereby achieving the axial sliding of the pushing member 682. When the pushing member 682 slides to a proper position, the button 656 is released, and the elastic member 658 is elastically reset and pushes the first clamping block 654 to move away from the second clamping block 655 until the engaging teeth 6544 of the first clamping block 654 are engaged to the racks 6117. The sliding member 650 is positioned to prevent the movable block 653 from sliding in the axial direction, thereby positioning the pushing member 682 in the axial direction. When it is required to rotate the pushing member 682, the button 656 is released, to cause the first clamping block 654 to move away from the second clamping block 655. The first rotating member 662 is rotated to drive the guide rails 651, the movable block 653 and the rotating post 664 to rotate. That is, the movable block 653 can rotate in the spaced enclosed by the first clamping groove 6548 of the first clamping block 654 and the second clamping groove 6558 of the second clamping block 655 about the axis of the pushing member 682, thereby driving the pushing member 682 to rotate with respect to the pushing tube 684. When it is required to rotate the pushing tube 684, the operating portion 6702 of the second rotating member 670 is rotated to cause the second rotating member 670 to rotate about the axis of the pushing member 682. As the proximal end of the pushing tube 684 is fixed in the through hole 6701 of the second rotating member 670, the pushing tube 684 rotates with the second rotating member 670.

The sliding means 65 of the pushing device 60 can drive the pushing member 682 to slide in the axial direction, so as to facilitate changing the position of the occluder in the blood vessel which is fixed to the distal end of the pushing member 682, and control the release speed of the occluder. After the occluder is moved by the pushing member 682 to a proper position, the button 656 is released, and the first clamping block 654 is moved towards the first shell 611 by the resetting force of the elastic member 658, so that the engaging teeth 6544 are engaged to the racks 6117, to achieve the positioning of the pushing member 682 and the occluder. Therefore, the occluder can be conveniently moved and positioned by the sliding means 65.

For the occluder fastening system, in an interventional operation, after a puncture position is determined and the puncture is completed, the distal end of the expansion rod 32 of the expander 30 is inserted into the sheath 23 from the proximal end of the sheath bendable adjustment device 20, and be exposed from the distal end of the sheath 23. As the distal end of the expander 30 gradually decreases in diameter to form a conical structure, the sheath can be conveniently leaded to the vicinity of a lesion site by an operator under an imaging device with the aid of the distal structure of the expander 30. After reaching the vicinity of the lesion site, the expander 30 is withdrawn, with the sheath 23 left in the body to establish a passage from the outside to the body. The distal end of the pushing component 68 of the pushing device 60 is caused to extend through the sealing seat 44 and the loading tube 42 of the loading component 40. Then the distal end of the pushing member 682 and the distal end of the pushing tube 684 fitted each other are detachably fixed to the occluder 10. The relative position of the pushing member 682 and the pushing tube 684 is changed to pull the occluder 10 in the axial direction, moving the pushing component 68 towards the proximal end to make the guide structure 111 of the occluder 10 be inserted in the loading tube 42, so that the occluder 10 can be easily received in the inner cavity of the loading tube 42 of the loading component 40. The distal end of the loading tube 42 is inserted into the sheath 23 to obtain a delivery system loaded with the occluder 10. The housing 61 of the pushing device 60 is held by a hand and the pushing component 68 is advanced towards the distal end, to deliver the occluder 10 to the distal end port of the sheath 23 of the sheath bendable adjustment device 20. By rotating the sheath bendable adjustment device 20 and bending the sheath 23 to align the sheath 23 with the rupture at the lesion site. The button 656 of the pushing device 60 is slowly pushed, to move the pushing component 68 towards the rupture to expose the occluder 10 from the sheath 23, and the occluder 10 is released to a proper position at the rupture. The button 656 of the pushing device 60 is moved to slowly move the pushing member 682 in the axial direction towards the proximal end, to make the proximal occluding portion 11 and the distal occluding portion 15 of the occluder 10 be located at two ends of the rupture. Under the actions of the sliding means 65, the first rotating means 66 and the second rotating means 670 of the pushing device 60, the occlusion and locking of the rupture by the occluder 10 and the release of the occluder 10 from the pushing member 682 and the pushing tube 684 are achieved. By this time, the first holding shoulder 116 of the proximal occluding portion 11 and the second holding shoulder 156 of the second occluding 15 well clamp the tissue surrounding the rupture, which facilitates endothelialization of the occlude.

The foregoing descriptions are embodiments of the present invention. It should be appreciated that some improvements and modifications can be made by those of ordinary skill in the art without departing from the spirit of the embodiments of the present invention, which should also be within the scope of the present application.

What is claimed is:

1. An occluder, comprising a proximal occlusion portion, characterized in that, the proximal occlusion portion has a proximal end provided with a first converged member, the proximal occlusion portion is provided with a conical guide structure on an outer periphery of the first converged member; and the guide structure has an inner diameter gradually decreasing from a distal end to a proximal end.

2. The occluder according to claim 1, wherein the occluder further comprises a distal occlusion portion and a waist arranged between the proximal occlusion portion and the distal occlusion portion, the proximal occlusion portion comprises a first annular groove extending to the waist and recessed towards the proximal end and a first side wall adjoining an outer edge of the first annular groove; the first side wall extends obliquely from the first annular groove towards the proximal end for a length and then turns to extend obliquely towards the distal end to be connected to a distal end of the guide structure; and wherein the first side wall and the guide structure define an annular valley recessed towards the distal end, and the first side wall and the first annular groove define a first holding shoulder.

3. The occluder according to claim 2, wherein the proximal occlusion portion is a structure woven by metal wires and having an internal space; the metal wires after woven which are adjacent to the first annular groove extend toward the proximal end at an angle of 30° to 45° with respect to an axial direction to form the first side wall, and then turn to extend towards the distal end to form the annular valley.

4. The occluder according to claim 2, wherein the distal occlusion portion comprises a second annular groove extending to the waist and recessed towards the distal end and a second side wall adjoining an outer edge of the second annular groove; the second side wall extends obliquely from the second annular groove towards the distal end and converges at a distal end of the distal occlusion portion; and wherein the second side wall and the second annular groove define a second holding shoulder, and the second holding shoulder and the first holding shoulder are arranged opposite to each other.

5. The occluder according to claim 4, wherein the distal occlusion portion is a structure woven by metal wires and having an internal space, the distal occlusion portion further comprises a second converged member located at the distal end; the metal wires after woven which are adjacent to the second annular groove extend towards the distal end to approach the second converged member at an angle of 30° to 45° with respect to the axial direction first to form the second side wall, and then further approach the second converged member and are fixed to the second converged member.

6. The occluder according to claim 4, wherein the first annular groove and the second annular groove are arranged oppositely at a proximal end and distal end of the waist.

7. The occluder according to claim 3, wherein the first converged member comprises an inner sleeve arranged at the proximal end of the proximal occlusion portion and a protrusion portion fixed to the inner sleeve; and wherein proximal ends of the metal wires are converged and positioned between the inner sleeve and the protrusion portion.

8. The occluder according to claim 7, wherein the protrusion portion is provided with a through hole in an axial direction, and wherein an external thread is provided on an outer wall of the protrusion portion.

9. The occluder according to claim 8, wherein the distal occlusion portion further comprises an outer sleeve and a connecting bolt connected to the outer sleeve; distal ends of the metal wires are converged and positioned between the outer sleeve and the connecting bolt; the connecting bolt extends towards the proximal end, an external thread is provided on an outer wall of the connecting bolt at a proximal end thereof; and the connecting bolt is capable of extending through the through hole and being screwed to a nut.

10. The occluder according to claim 9, wherein a thread direction of the external thread of the connecting bolt is opposite to a thread direction of the external thread of the protrusion portion.

11. The occluder according to claim 9, wherein one end of the connecting bolt adjacent to the outer sleeve is provided with a protruding plate in a radial direction; and the protruding plate has an outer diameter larger than an inner diameter of the through hole.

12. An occluder fastening system, comprising a sheath bendable adjustment device, a loader, a pushing device, and an occluder according to claim 1, wherein the sheath bendable adjustment device comprises a sheath; the loader comprises a loading tube, wherein a distal end of the loading tube is inserted in the sheath; and the pushing device comprises a pushing component, and the pushing component is capable of withdrawing the occluder into the sheath.

13. The occluder fastening system according to claim 12, wherein the pushing component comprises a pushing member, a pushing tube movably mounted around the pushing member, and a nut positioned inside a distal end of the pushing tube; and wherein a distal end of the pushing member is detachably connected to the connecting bolt of the occluder, and an inner wall of the pushing tube at a distal end is provided with an internal thread corresponding to the external thread of protrusion portion.

14. The occluder fastening system according to claim 13, wherein the pushing tube comprises a body section and an extension section arranged at a distal end of the body section; the nut is capable of being positioned inside the extension section; the internal thread is arranged on an inner wall of the extension section at a distal end thereof; and the internal thread is capable of fitting with the outer thread of the protrusion portion.

15. The occluder fastening system according to claim 14, wherein a thread screwing direction between the extension section and the protrusion portion is opposite to a thread screwing direction between the connecting bolt and the nut.

16. The occluder fastening system according to claim 14, wherein the extension section and the protrusion portion have a thread number and thread a pitch equaling to or greater than a thread number and a thread pitch of the nut, to make a proximal end of the connecting bolt extend out of a proximal end surface of the nut.

17. The occluder fastening system according to claim 14, wherein an outer diameter of the body section is smaller than an outer diameter of the extension section, and an inner diameter of the body section is smaller than an inner diameter of the extension section, such that a step surface is formed in the pushing tube between a proximal end of the extension section and a distal end surface of the body section, and the nut is capable of abutting against the step surface.

18. The occluder fastening system according to claim 17, wherein an inner wall of the extension section at the proximal end is provided with an engaging groove, the nut is engaged in the engaging groove, the engaging groove prevents the nut from rotation in the pushing tube, and the nut is axially slidable in the extension section.

19. The occluder fastening system according to claim 13, wherein an outer wall of the pushing member at the distal end is provided with an external thread corresponding to the screw hole of the connecting bolt, and the distal end of the pushing member is configured to movably extend through the nut and the extension section.

20. The occluder fastening system according to claim 13, wherein the pushing device comprises a casing with an accommodating space, a first pushing component partially received in the casing, and a second pushing component partially mounted around the casing, wherein the first pushing component comprises a sliding means and a first rotating means, the sliding means is configured to drive the pushing member to slide axially and be positioned in the pushing tube, and the first rotating mechanism is configured to drive the pushing member to rotate in the pushing tube; and wherein the second pushing component comprises a second rotating mechanism, and the second rotating mechanism is configured to drive the pushing tube to rotate outside the pushing member.
